(19) United States
(12) Patent Application Publication
(10) Pub. No.: US 2024/0108854 A1
(43) Pub. Date: Apr. 4, 2024

(54) CATHETER WITH DISTAL BRAID TERMINATIONS

(71) Applicant: Neuravi Limited, Gatway (IE)

(72) Inventors: Karl KEATING, Galway (IE); Paulo RATOLA, Galway (IE)

(73) Assignee: Neuravi Limited, Galway (IE)

(21) Appl. No.: 17/955,930

(22) Filed: Sep. 29, 2022

(51) Int. Cl. A61M 25/00

(52) U.S. Cl. CPC A61M 25/0053; A61M 25/001; A61M 25/0045; A61M 2205/0266; A61M 2207/10

(57) ABSTRACT

An aspiration catheter having a braid formed from a continuous braiding process and having a braided distal tip design with braid terminations joining strand ends is presented herein. The braid terminations can include sleeves, bands, crimps, or other types of connectors that secure pairs ends of oppositely-wound strands to each other at a distal circumference of the braid. The braid terminations can include radiopaque material to help visualize the distal end of the catheter during a procedure.

### FIELD OF INVENTION

The present invention generally relates devices and methods for removing acute blockages from blood vessels during intravascular medical treatments. More specifically, the present invention relates to retrieval catheters with expandable tips into which an object or objects can be retrieved.

### BACKGROUND

Clot retrieval aspiration catheters and devices are used in mechanical thrombectomy for endovascular intervention, often in cases where patients are suffering from conditions such as acute ischemic stroke (AIS), myocardial infarction (MI), and pulmonary embolism (PE). Accessing the neurovascular bed in particular is challenging with conventional technology, as the target vessels are small in diameter, remote relative to the site of insertion, and highly tortuous. These catheters are frequently of great length and must follow the configuration of the blood vessels in respect of all branching and windings. Traditional devices are often either too large in profile, lack the deliverability and flexibility needed to navigate particularly tortuous vessels, or are not effective at removing a clot when delivered to the target site.

Many existing designs for aspiration retrieval catheters are often restricted to, for example, inner diameters of 6Fr or between approximately 0.068-0.074 inches. Larger sizes require a larger guide or sheath to be used, which then necessitates a larger femoral access hole to close. Most physicians would prefer to use an 8Fr guide/6Fr sheath combination, and few would be comfortable going beyond a 9Fr guide/7Fr sheath combination.

In many existing aspiration catheters, the diameter of the catheter is consistent along the length of the catheter to the distal tip of the catheter. This means that once at the target site, a clot can often be larger in size than the inner diameter of the aspiration catheter and must otherwise be immediately compressed to enter the catheter mouth. This compression can lead to bunching up and subsequent shearing of the clot during retrieval. Firm, fibrin-rich clots can also become lodged in the fixed-mouth tip of these catheters making them more difficult to extract. This lodging can also result in shearing where softer portions breaking away from firmer regions of the clot.

Some aspiration catheters include beveled openings to provide an oval shaped opening to attempt to mitigate the likelihood of clot shearing. Aspiration catheters have been proposed which include expandable distal ends, which may or may not have beveled openings, to further mitigate the likelihood of clot shearing.

In many examples, the fixed-mouth catheters and those with expandable distal tips can have an underlying braid as the primary supporting backbone. Forming the distal end of the braid of the catheter to accomplish needs during a treatment while also having a scalable manufacturing process is a desirable.

### SUMMARY

The present disclosure is generally directed to an aspiration catheter having a braid formed from a continuous braiding process and having a braided distal tip design with braid terminations joining strand ends. The braid terminations can include sleeves, bands, crimps, or other types of connectors that secure pairs ends of oppositely-wound strands to each other at a distal circumference of the braid. The braid terminations can include radiopaque material to help visualize the distal end of the catheter during a procedure.

An example catheter can include a braid, a plurality of braid terminations, an inner liner, and an outer jacket. The braid can extend through a distal section of the catheter and can include clockwise strands and counter-clockwise strands. The plurality of braid terminations can each join a distal portion of a clockwise strand to a distal portion of a counter-clockwise strand. The inner liner can be disposed within the braid. The outer jacket can be disposed over the braid.

An inner diameter of the distal section of the catheter can be expandable when the distal section is unconstrained and compressible when the distal section is constrained.

The plurality of braid terminations can each include a sleeve over the distal portion of the clockwise strand and over the distal portion of the counter-clockwise strand.

The sleeve can include a crimped B-shape having a flat surface and two curved surfaces opposite the flat surface. The sleeve can further include ends terminating the curved surfaces between the distal portion of the clockwise strand and the distal portion of the counter-clockwise strand.

The sleeve can include a tubular shape having a lumen therethrough. The distal portion of the clockwise strand and the distal portion of the counter-clockwise strand can extend into the lumen. The sleeve can have an outer wall and an inner wall. The outer wall can be thicker than the inner wall.

The distal portion of the clockwise strand and the distal portion of the counter-clockwise strand can each be oriented parallel to a longitudinal axis defined by the distal section of the catheter.

The distal portion of the clockwise strand and the distal portion of the counter-clockwise strand can each be oriented orthogonal to a longitudinal axis defined by the distal section of the catheter.

The plurality of braid terminations can include a band joining a first pair of braid distal portions, joining a second pair of braid distal portions, and extending between the first pair of braid distal portions and the second pair of braid distal portions. Each pair of distal portions can include a distal portion of a clockwise strand and a distal portion of a counter-clockwise strand.

The plurality of braid terminations can each include radiopaque material.

The braid can have a greater braid pattern density in a proximal section of the catheter compared to a braid pattern density in the distal section of the catheter.

The clockwise strands and/or the counter-clockwise strands can include radiopaque material. The clockwise strands and/or the counter-clockwise strands can include memory shape material.

The plurality of braid terminations can each include a laser weld.

The plurality of braid terminations can each include adhesive.

An example method of constructing a catheter can include some or all of the following steps which can be executed in a variety of orders. The method can also include additional steps, including those yet to be developed, as understood by a person skilled in the pertinent art. The method can include braiding clockwise strands and counter-clockwise strands directly over a catheter liner disposed over a spool to form a braid, joining a distal portion of a clockwise strand to a distal portion of a counter-clockwise strand by a braid termination for some or all of the clockwise strands and counter-clockwise strands, and heat-setting a distal segment of the braid to a larger diameter compared to a diameter of a proximal segment of the braid.

Joining the distal portion of the clockwise strand to the distal portion of the counter-clockwise strand by the braid termination for some or all of the clockwise strands and counter-clockwise strands can further include crimping a sleeve around the distal portion of the clockwise strand and the distal portion of the counter-clockwise strand.

Joining the distal portion of the clockwise strand to the distal portion of the counter-clockwise strand by the braid termination for some or all of the clockwise strands and counter-clockwise strands can further include sliding a sleeve over the distal portion of the clockwise strand and the distal portion of the counter-clockwise strand.

The method can further include aligning the distal portion of the clockwise strand and the distal portion of the counter-clockwise strand parallel to a longitudinal axis of the catheter.

The method can further include aligning the distal portion of the clockwise strand and the distal portion of the counter-clockwise strand orthogonal to a longitudinal axis of the catheter.

Braiding clockwise strands and counter-clockwise strands directly over the catheter liner disposed over the spool to form the braid can further include braiding clockwise strands and counter-clockwise strands to have a greater braid pattern density on a proximal segment of the braid compared to a braid pattern density on the distal segment of the braid.

The method can further include joining the distal portion of the clockwise strand to the distal portion of the counter-clockwise strand by the braid termination for some or all of the clockwise strands and counter-clockwise strands while the braid is directly over the catheter liner disposed over the spool.

The plurality of braid termination can include a laser weld.

The plurality of braid termination can include adhesive.

### BRIEF DESCRIPTION OF THE DRAWINGS

The above and further aspects of this invention are further discussed with reference to the following description in conjunction with the accompanying figures, in which like numerals indicate like structural elements and features in various figures. Drawings are not necessarily to scale, emphasis instead being placed upon illustrating principles of the invention. The figures depict one or more implementations of the inventive devices, by way of example only, not by way of limitation. It is expected that those of skill in the art can conceive of and combine elements from multiple figures to better suit the needs of the user.

FIG. 1 is an illustration of a distal portion of a catheter including an expandable distal segment having a braid and braid terminations according to aspects of the present invention.

FIG. 2A is an illustration of the distal portion of the catheter during a manufacturing step in which a pair of braid ends are positioned to facilitate formation of a braid termination according to aspects of the present invention.

FIG. 2B is an illustration of the distal portion of the catheter during a manufacturing step in which a sleeve is attached to the pair of braid ends according to aspects of the present invention.

FIG. 2C is an illustration of the distal portion of the catheter during a manufacturing step after which excess distal extensions of the strands are removed according to aspects of the present invention.

FIG. 3A is an illustration of a sleeve of a braid termination according to aspects of the present invention.

FIG. 3B is an illustration of a first example braid termination over a liner according to aspects of the present invention.

FIG. 4 is an illustration of a second example braid termination over a liner according to aspects of the present invention.

FIG. 5 is an illustration of a third example braid termination over a liner according to aspects of the present invention.

FIG. 6A is an illustration of a fourth example braid termination over a liner according to aspects of the present invention.

FIG. 6B is an illustration of a distal portion of a braid including braid terminations configured similar to the fourth example braid terminations over the liner according to aspects of the present invention.

FIG. 6C is an illustration of a distal portion of a catheter including the braid illustrated in FIG. 6B according to aspects of the present invention.

FIG. 7A is a perspective view illustration of a distal portion of a braid including fifth example braid terminations including bands each joining two pairs of strand ends according to aspects of the present invention.

FIG. 7B is a profile view illustration of the braid illustrated in FIG. 7A, wherein the view includes a longer segment of the braid compared to FIG. 7A according to aspects of the present invention.

FIG. 8 is an illustration of a braid having sections of differing braid pattern density according to aspects of the present invention.

FIGS. 9A through 9C are illustrations of manufacturing steps of an example catheter according to aspects of the present invention.

FIG. 10 is a flow diagram of a method of constructing an example catheter according to aspects of the present invention.

FIG. 11 is an illustration of a procedure utilizing an example catheter according to aspects of the present invention.

### DETAILED DESCRIPTION

The invention is not necessarily limited to the examples described, which can be varied in construction and detail. The terms “distal” and “proximal” are used throughout the preceding description and are meant to refer to a positions and directions relative to a treating physician. As such, “distal” or distally” refer to a position distant to or a direction away from the physician. Similarly, “proximal” or “proximally” refer to a position near or a direction towards the physician. Furthermore, the singular forms “a,” “an,” and “the” include plural referents unless the context clearly dictates otherwise.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 71% to 99%.

When used herein, the terms “tubular” and “tube” are to be construed broadly and are not limited to a structure that is a right cylinder or strictly circumferential in cross-section or of a uniform cross-section throughout its length. For example, the tubular structure or system is generally illustrated as a substantially right cylindrical structure. However, the tubular system may have a tapered or curved outer surface without departing from the scope of the present invention.

Some design and manufacturing considerations for braid-supported catheters can include, but are not limited to, effectively bonding layers, meeting the flexibility criteria for aspiration procedures, having an atraumatic distal end for use in fragile vessels without causing substantial trauma, having sufficient hoop strength at the distal portion of the catheter to resist aspiration forces without collapse, having a structure capable of folding down consistently and repeatably when advanced, or retrieved through an outer guide and/or sheath, and/or smaller blood vessel, and/or being capable of expanding elastically when deployed distally through an outer guide and/or sheath and/or as a clot is ingested for better interaction with and retention of the clot.

In some procedures, it can be desirable for the distal braid ends to be atraumatic to mitigate risk of a cut braid end traumatizing a vessel. The distal end of the braid can be covered by a polymer catheter tip; however, it is desirable for the polymer tip to be flexible and itself atraumatic. A distal braid end that is atraumatic to mitigate likelihood of braid ends breaking through or deforming the polymer tip without significantly increasing manufacturing cost or complexity is desired. An example catheter is presented herein with these design considerations in mind.

A braided tip design to facilitate manufacturability of a catheter having a traumatic distal braid is presented herein. The catheter can also include radiopaque in the braided tip. Some examples include an axial sleeve radiopaque braid termination. Some examples include a circumferential sleeve radiopaque braid termination. Some examples include welded marker band segment braid terminations. Some examples include a continuous braiding process.

The braid need not be formed in separate pieces, and the catheter can include one continuous braid. The braid transfer process can be easier for a continuous braid compared to a braid transfer process of a braid formed in segments. A continuous braid catheter manufacturing process can reduce manufacturing cost compared to a segmented braid catheter manufacturing process. Higher braid pattern density (higher pics per inch, PPI) can potentially be more easily achieved for a continuous braid which does not have to move from one mandrel to another during manufacturing compared to a braid segment which is moved from one mandrel to another during manufacturing. Moving a braid from one mandrel to another during manufacturing can be challenging if the braid grips the first mandrel too tightly to allow transfer. This can be overcome by annealing or heat setting the wire to relax the dislocations in the material structure; however, it can be beneficial to keep the wires in the worked state to prevent making the material more ductile through annealing.

The designs herein can be utilized for a super-bore clot retrieval catheter with a large internal lumen and a distal funnel tip that can self-expand to a diameter larger than that of the guide or sheath through which it is coaxially delivered. The designs can have a proximal elongate body for the shaft of the catheter, and a distal tip with an expanding braided support structure and outer polymeric jacket to give the tip atraumatic properties. The braided support can be designed so that the expansion capability is variably focused within an axial portion of the tip section. The braid cells can be capable of easily and repeatably collapsing for delivery and expanding for good clot reception and resistance under aspiration. Sections of the tip can have the ability to further expand beyond the free shape of the expanded deployed configuration when ingesting a clot. The catheter's braid and tip designs can be sufficiently flexible to navigate highly tortuous areas of the anatomy and be able to recover its shape to maintain the inner diameter of the lumen when displaced in a vessel.

Accessing the various vessels within the vascular, whether they are coronary, pulmonary, or cerebral, involves well-known procedural steps and the use of a number of conventional, commercially-available accessory products. These products, such as angiographic materials, mechanical thrombectomy devices, microcatheters, and guidewires are widely used in laboratory and medical procedures. When these products are employed in conjunction with the devices and methods of this invention in the description below, their function and exact constitution are not described in detail. Additionally, while the description is in many cases in the context of thrombectomy treatments in intercranial arteries, the disclosure may be adapted for other procedures and in other body passageways as well.

Specific examples of the present invention are now described in detail with reference to the Figures, where identical reference numbers indicate elements which are functionally similar or identical.

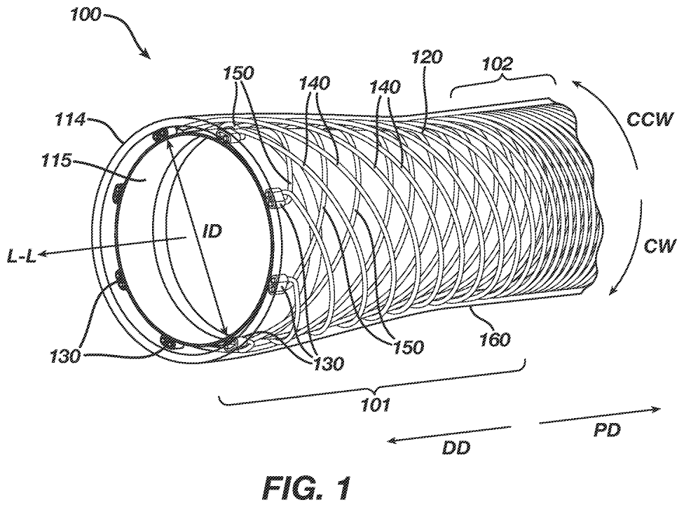

FIG. 1 is an illustration of a distal portion of a catheter 100 including an expandable distal section 101 having a braid 120, braid terminations 130, an inner liner 115, and an outer jacket 160. The outer jacket 120 extends to a distal end 114 of the catheter 100, distal of the braid terminations 130. The braid 120 includes counter-clockwise braid strands 140 and clockwise braid strands 150 that are woven together to form the braid 120. The counter-clockwise braid strands 140 curve in a counter-clockwise direction CCW in a distal direction DD and viewed from the distal end 114 of the catheter 100, and the clockwise braid strands 150 curve in a clockwise direction CW in a distal direction DD and viewed from the distal end 114 of the catheter 100. The distal portion of the catheter 100 can define a longitudinal axis L-L.

The distal portion of the catheter illustrated in FIG. 1 also includes a proximal or intermediate section 102 of the catheter 100 located in a proximal direction PD in relation to the distal section 101. The distal section 101 can be flared, having a larger inner diameter ID, at least at the distal end 114, in comparison to the proximal or intermediate section 102. The inner diameter ID can be expandable when the distal section 101 is unconstrained. The proximal or intermediate section 102 can have a greater braid pattern density (i.e. higher PPI, higher braid angle) compared to a braid pattern density of the distal section 101.

The illustrated catheter 100 includes a funnel tip design that can be configured to collapse and expand during use. A distal end of a clockwise braid strand 150 that is fixed at a braid termination 130 cannot move apart from a distal end of a counter-clockwise braid strand 140 fixed at the same braid termination 130. This can reduce the likelihood that a braid end can move to a position that protrudes through the outer jacket 120, compared to a similar braid lacking the braid terminations 130. Movement of the strands 140, 150 during radial expansion/compression of the tip 114 or through lateral bending of the catheter 100 when navigating tortuous vessels can apply forces to the braid strands 140, 150, and the terminations 130 can inhibit the braid strands 140, 150 from protruding through the outer jacket 120 as these forces are on the braid strands 140, 150.

The outer jacket 160 can include one or more layers and can vary in composition over a length of the catheter 100. In some examples, the outer jacket 160 can be more pliable in the distal section 101 of the catheter compared to more proximal sections 102 of the catheter 100.

The braid terminations 130 can include radiopaque material. Visibility of the distal end 114 of the catheter 100 can be further enhanced by mounting on a composite wire such as NiTi DFT with 10-40% Platinum or Tantalum fill distal of the braid, under the outer jacket 160. The presence of 60-90% NiTi tube over a 10-40% Platinum core can allow the braid 120 to have shape memory properties while also having visibility under fluoroscopy. NiTi DFT 20 Pt strands 140, 150 can give the denser braid pattern (100 to 170 PPI) of the proximal portion 102 shaft visibility, however, where the braid 120 density decrease to allow for tip collapsibility of a funnel design, added radiopacity may be desirable. Radiopaque braid terminations 130 can give visibility of the distal tip 114 of the catheter 100. A short length of Tungsten filled radiopaque jacket material can be added to enhance visibility at the distal end 114 of the catheter 100.

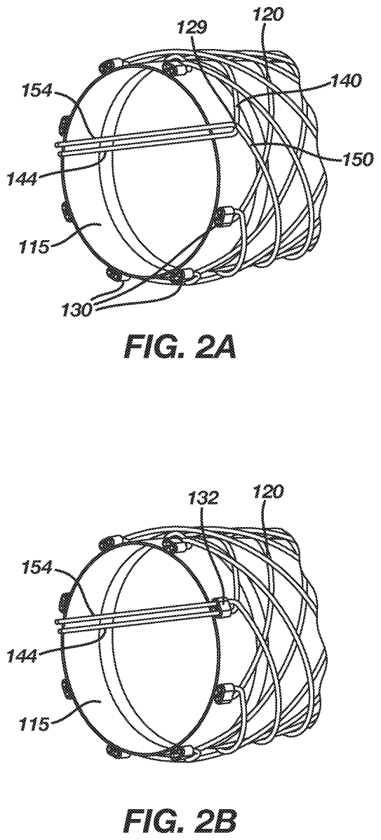

FIG. 2A is an illustration of the distal portion of the catheter 100 during a manufacturing step in which a pair of distal portions 154, 144 of strands 140, 150 are positioned to facilitate formation of a braid termination 130.

FIG. 2B is an illustration of the distal portion of the catheter 100 during a manufacturing step in which a sleeve 132 is attached to the pair of distal portions 144, 154 of strands 140, 150. The sleeve 132 can be crimped to hold the distal portions 144, 154 together. Additionally, or alternatively, the braid terminations 130 can include adhesive, solder, and/or laser welding to join the distal portions 144, 154 and/or secure the sleeve 132 in place. The sleeve 132 can be made of radiopaque material to facilitate visualization of the distal portion of the catheter 100 near the distal end 114.

FIG. 2C is an illustration of the distal portion of the catheter during a manufacturing step after which excess distal extensions of the distal portions 144, 154 of the strands 140, 150 are removed.

The braid 120 can include a first segment 121 that extends in the proximal direction PD from a distal end of the braid 120, and the braid 120 can include a second segment 122 that extends in a proximal direction from the first segment 121. The first braid segment 121 can be positioned in the distal section 101 of the catheter. The second braid segment 122 can be positioned in the proximal or intermediate section of the catheter 102. The second braid segment 122 can have a greater braid pattern density than the first braid segment 121.

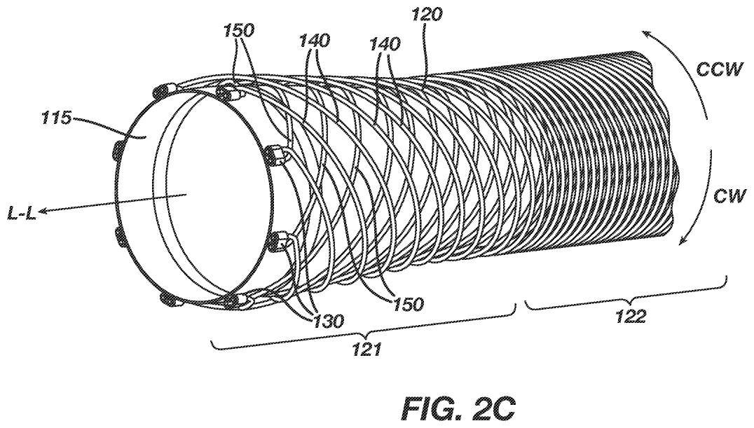

FIG. 3A is an illustration of a first example sleeve 132 of a first example braid termination 130. The first example sleeve 132 has a B-shape. The B-shape has a flat surface 134 and two curled surfaces 135 opposite the flat surface 134. The sleeve 132 also has two ends 133 terminating the curled surfaces 135.

FIG. 3B is an illustration of the first example braid termination 130 over a liner 115. The first example sleeve 132 is oriented such that the flat surface 134 is facing outward and the curved surfaces 135 are facing inward such that the flat surface 134 faces the jacket 160 and the curved surfaces 135 faces the liner 115. Alternatively, the flat surface 134 can face inward and the curved surfaces 135 can face outward. The ends 133 of the sleeve 132 are positioned between the distal portion 153 of the clockwise strand 150 and the distal portion 143 of the counter-clockwise strand 140.

The clockwise strand 150 and the counter-clockwise strand 140 can overlap each other at a distal braid junction 129. The distal braid junction 129 is positioned in a proximal direction in relation to the sleeve 132. The distal braid junction 129 can be a position at which the clockwise strand 150 and the counter-clockwise strand 140 would otherwise overlap where it not for the sleeve 132, following the braiding pattern of the first braid segment 121. The distal portions 143, 153 of the strands 140, 150 are parallel to a longitudinal axis L-L (FIG. 1) defined by the distal portion of the catheter 100. Strand ends 142, 152 are oriented on a distal side of the B-shaped sleeve 132.

The B-shaped sleeve 132 can be produced by folding flat sheet material or through extrusion. Sleeve 132 may be of a material similar to braid 120 to allow easy welding to fix position. More preferably, the sleeve 132 is made from a radiopaque material such as Platinum Iridium or Tungsten so that the distal tip 114 of the catheter is highly visible if mounted on a SS or NiTi braid 120.

The B-shaped sleeve 132 can have a width of about 0.0005″ (inches) to about 0.002″ with a length of about 0.006″ to about 0.020″, more preferably a width of about 0.001″ to about 0.0015″ with a length of about 0.008″ to about 0.012″. The sleeve 132 can be slid over strands 140, 150 after cutting the strands 140, 150 from a continuous spool or crimped over strands 140, 150 before cutting the strands 140, 150 from a continuous spool. The B-shaped sleeve 132 can be fixed in position through crimp, adhesive, weld, and/or solder.

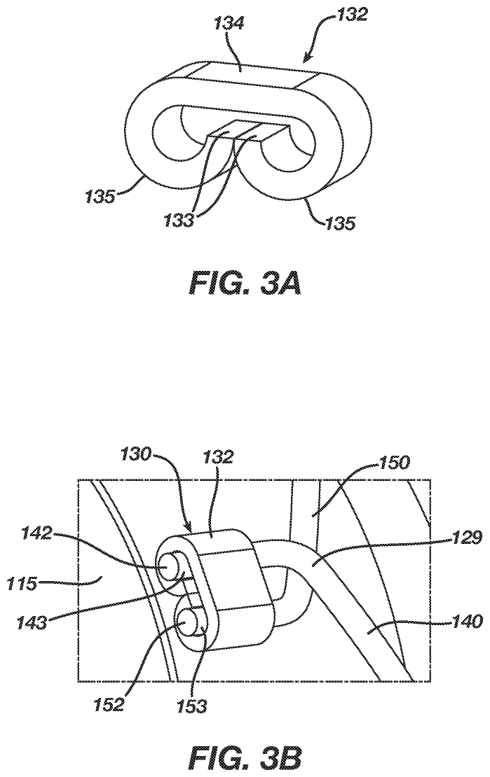

FIG. 4 is an illustration of a second example braid termination 230 over a liner 115. The second example braid termination 230 includes a second example sleeve 232 having a tubular shape with a lumen 236 therethrough. The distal portion 153 of the clockwise strand 150 and the distal portion 143 of the counter-clockwise strand 140 extend into the lumen 236. The distal portions 143, 153 of the strands 140, 150 are parallel to a longitudinal axis L-L (FIG. 1) defined by the distal portion of the catheter 100. Strand ends 142, 152 are oriented on a distal side of the O-shaped sleeve 232.

The sleeve 232 has a flat outer wall 234 and a flat inner wall 235. The O-shaped sleeve 232 can have a width of about 0.0005″ to about 0.002″ with a length of about 0.006″ to about 0.020″, more preferably a width of about 0.001″ to about 0.0015″ with a length of about 0.008″ to about 0.012″. The O-shaped sleeve 232 can be slid over distal segments 143, 153 of strands 140, 150 after the strands 140, 150 are cut from a continuous spool. The O-shaped sleeve 232 can be fixed in position through crimp, adhesive, weld, and/or solder.

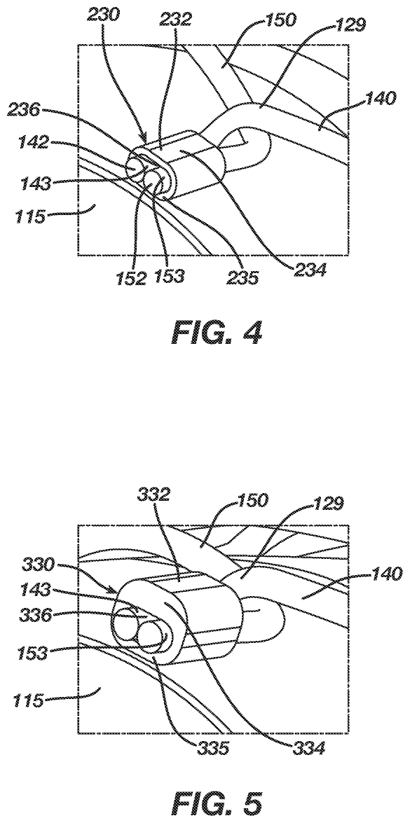

FIG. 5 is an illustration of a third example braid termination 330 over a liner 115. The third example braid termination 330 includes a third example sleeve 332 having a tubular shape with a lumen 336 therethrough. The distal portion 153 of the clockwise strand 150 and the distal portion 143 of the counter-clockwise strand 140 extend into the lumen 336. The distal portions 143, 153 of the strands 140, 150 are parallel to a longitudinal axis L-L (FIG. 1) defined by the distal portion of the catheter 100. Strand ends 142, 152 are oriented on a distal side of the variable thickness O-shaped sleeve 132.

The sleeve 332 has a flat outer wall 334 and a flat inner wall 335. The outer wall 334 is thicker than the inner wall 335. The variable thickness O-shaped sleeve 332 has a variable wall thickness to enhance visibility under fluoroscopy. The variable thickness O-shaped sleeve 332 can be slid over distal segments 143, 153 of strands 140, 150 after the strands 140, 150 are cut from a continuous spool. The variable thickness O-shaped sleeve 232 can be fixed in position through crimp, adhesive, weld, and/or solder.

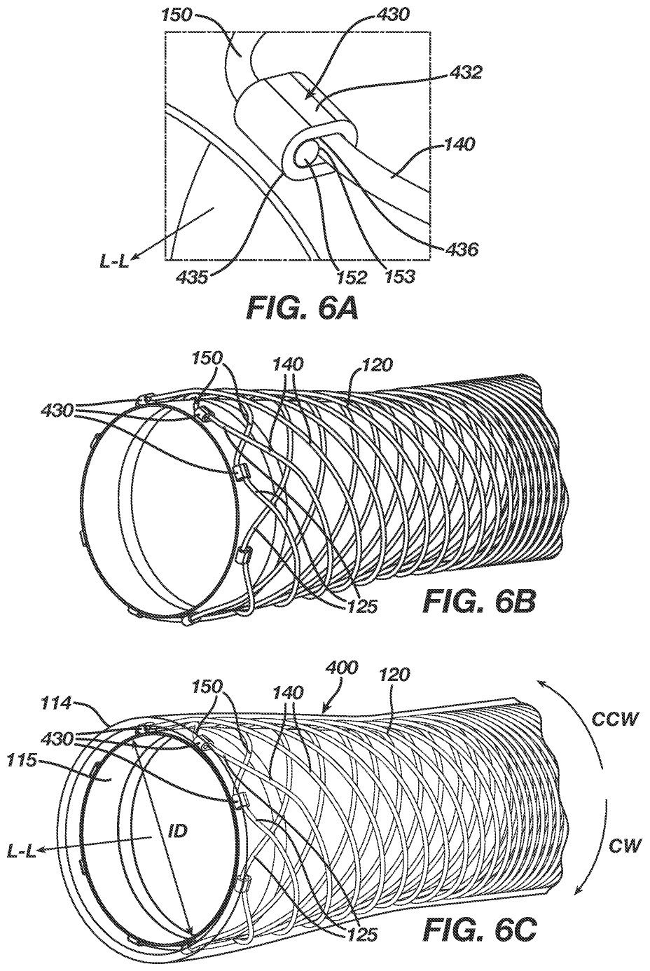

FIG. 6A is an illustration of a fourth example braid termination 430 over a liner 115. The fourth example braid termination 430 includes a fourth example sleeve 432 having a tubular shape with a lumen 436 therethrough. The distal portion 153 of the clockwise strand 150 and the distal portion 143 of the counter-clockwise strand 140 extend into the lumen 436. The strand ends 152, 142 are oriented on opposite sides of the sleeve 432 (a clockwise side of the sleeve 432 and a counter-clockwise side of the sleeve 432). The distal portions 143, 153 of the strands 140, 150 are orthogonal to a longitudinal axis L-L (FIG. 1) defined by the distal portion of the catheter 100. The sleeve 432 has a flat outer wall 434 and a flat inner wall 435.

FIG. 6B is an illustration of a distal portion of a braid 120 including braid terminations 430 configured similar to the fourth example braid terminations 430 over the liner 115. The braid 120 illustrated in FIG. 6B is otherwise configured similarly to the braid 120 illustrated in previous figures.

Sleeve designs can be orientated with openings that are circumferential such that the openings fall in closer alignment with the direction of the braid strands 140, 150 thus allowing for an atraumatic finish to the braid terminations 430.

The braid terminations 430 form distal hoops 125 similar to a design in which each braid strand is folded back at the distal braid end so that each strand has a clockwise segment and counter-clockwise segment extending from the braid distal end 114 as described in U.S. patent application Ser. No. 17/518,428 filed Nov. 3, 2021, and incorporated by reference herein as if set forth in its entirety (see hoops 230 illustrated throughout U.S. patent application Ser. No. 17/518,428). The hoops 125 illustrated in FIG. 6B can reduce the likelihood of braid ends 142, 152 migrating through polymer encapsulation of the outer jacket 160 and other polymer encapsulation at the distal end 114 of the catheter 100 during use. The braid terminations 430 can also allow the braid 120 to be more easily formed continuously compared to a braid having strands that are folded back at the distal end.

FIG. 6C is an illustration of a distal portion of a catheter including the braid 120 illustrated in FIG. 6B, braid terminations 430 configured similarly to the fourth example braid terminations 430, the inner liner 115, and an outer jacket 160 configured similarly to the outer jacket 160 illustrated in FIG. 1. The braid 120 can be woven over the inner liner 115, and the outer jacket 160 can be applied over the braid 120 and inner liner 115.

The atraumatic design of the fourth example braid terminations 430 may reduce likelihood of protrusion of strand ends 142, 152 through very low durometer outer jackets 160 such as Neusoft 42A, RezAlloy 40A, Chronoprene 40A, and the like.

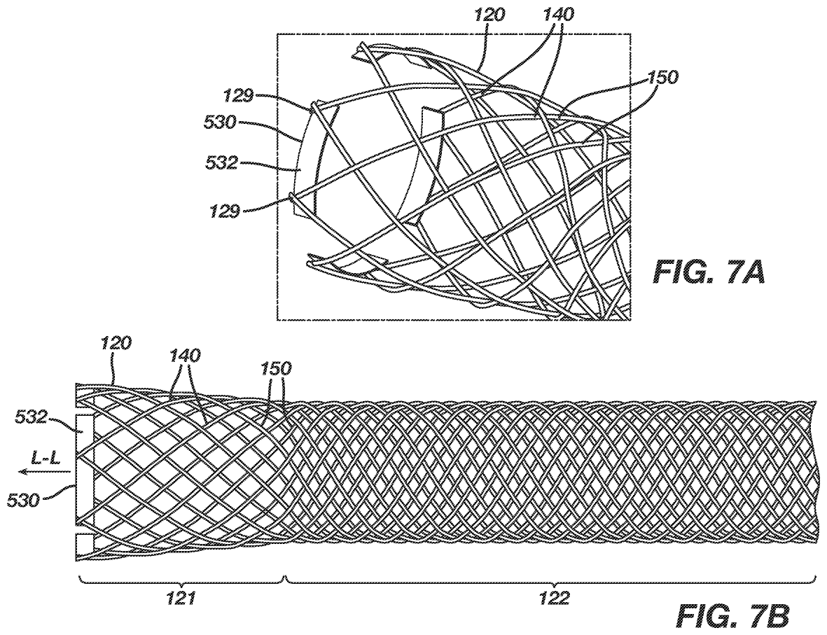

FIG. 7A is a perspective view illustration of a distal portion of a braid 120 and fifth example braid terminations 530 including bands 532 each joining two pairs of strand ends near distal braid junctions 129. The bands 532 are preferably radiopaque.

As illustrated, the distal portion of the braid 120 can include eight clockwise strands 150 and eight counter-clockwise strands 140 forming eight pairs of strand ends near eight distal braid junctions 129. As illustrated, the catheter can include four bands 532 each joining two pairs of strands 140, 150 near distal braid junctions 129.

The catheter 100 can include an even number of clockwise strands 150, the same even number of counter-clockwise strands 140, and a number of bands 532 that is half the even number of strands 140, 150 such that each band 532 joins two pairs of strand ends. Alternatively, the catheter 100 can include a number of clockwise strands 150 that is divisible by three, the same number of counter-clockwise strands 140, and a number of bands 532 that is one-third of the number of strands 140, 150 such that each band 532 joins three pairs of strand ends. Alternatively, the catheter can include a mix of bands that join only two pairs of strand ends, and bands that join only three pairs of strand ends. The catheter 100 can include three bands 532, four bands 532, five bands 532 for most catheter dimensions disclosed herein. The catheter 100 can include more bands 532 for higher density braids 120 and/or larger diameter catheters as understood by a person skilled in the pertinent art.

A round, or preferably flat, braid 120 can be woven over a liner 115 through a continuous braid process. A flat braid will be easier to laser weld to the marker band due to the increase in surface area contact, however, the ends of a round braid could be flattened with a press tool to aid the laser welding process. The distal segment 121 of the braid 120 can be flared through heat set or annealing step. The braid 120 can be cut near the distal braid junctions 129. The bands 532 can be laser welded to the distal portions of the braid strands 140, 150 prior to reflow.

FIG. 7B is a profile view illustration of the braid illustrated in FIG. 7A, wherein the view includes a longer segment of the braid compared to FIG. 7A.

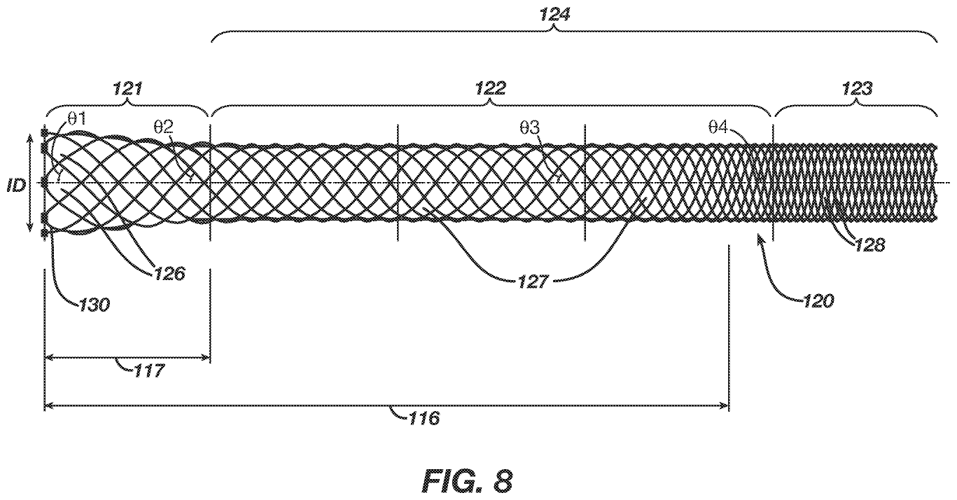

FIG. 8 is an illustration of a braid 120 divided into sections having decreasing braid pattern density, PPI, and braid angles in the distal direction DD. The braid can include sections that have substantially uniform braid pattern density within those sections and/or the braid can include sections that have a gradual transition of braid pattern density. As illustrated, the braid 120 includes a first, distal segment 121 that has the lowest braid pattern density, an intermediate section 122 that has a transitional braid pattern density, and a most proximal section 123 having the highest braid pattern density of those illustrated. The distal segment has a low braid angle θ1 near a distal end of the braid 120, and the braid angle θ2 may increase proximally. The intermediate segment can have a transition of braid angles θ2, θ3, θ4 that increase proximally. The braid pattern density and braid angle θ4 of the most proximal section 123 can be maintained to a proximal end of the braid 120, can increase proximally to facilitate pushing of the catheter, or can vary according to other design considerations as understood by a person skilled in the pertinent art.

The braid 120 can further be considered to have sections based on inner diameter ID. The distal segment 121 of the braid 120 can be pre-formed to a funnel shape having an inner diameter ID that tapers in the proximal direction. The proximal section 124 of the braid 120 can include the intermediate section 122 and the most proximal section 123 and can have a substantially uniform inner diameter ID.

Sixteen Nitinol wires in a one over one half-diamond pattern can be joined at braid terminations 130 at a distal end of the braid 120. Several variations of the braid pattern are possible and can be adapted to include the braid terminations 130 as understood by a person skilled in the pertinent art. For instance, a herringbone braid pattern with one wire extending over two wires and under two wires may provide better flexibility in comparison to the one wire under one over one wire half diamond pattern. Another option is a full diamond pattern in which two wires extend together under a group of two and over a group of two wires, which may provide more hoop strength. Another option is to reduce the number of hoops at the distal end to four or six. Another option is to increase the number of hoops at the distal end to ten or twelve. More wires may provide more stiffness and more compressive strength while fewer wires may provide better flexibility but less compressive strength.

Generally, the PPI of the braid can have an inverse relationship with the expandability of a particular section of the braid 120. The distal expansile segment 121 of the braid can have a PPI that decreases in the distal direction allowing for greater ability to expand at the distal end of the braid 120. The second, intermediate section 122 can have a PPI that decreases in the distal direction. Regions with a larger PPI can have greater flexibility but limited expansion capability. Similarly, regions with lower PPI values can trade hoop strength for markedly greater expansion capability. The second, intermediate section 122 can be configured to have some ability to expand to accommodate clot ingestion, with expansion that is preferably less than the distal segment 121. The ability of the intermediate section 122 to expand can be decreased in the proximal direction to aid in compressing an ingestion clot as the clot moves proximally through the catheter 100. The braid in a most proximal section 123 can be configured to facilitate pushing of the catheter 100 through vasculature and can be configured to resist expansion as a clot passes therethrough.

The distalmost cells 126 can have the smallest cell angle corresponding to the greatest expansion capability. The distalmost cells can have a cell angle of about 65° to about 125°, and more preferably about 110°. The braid angles of the distal expansile segment 121 can increase proximally to about 130° over the length 117 of this segment 121. The intermediate section 122 can have a tubular profile with a gradual progressive increase in cell angle proximally (for example from about 105° distally (preferably 130°) and increasing to about 154° proximally) and PPI (from about 40° distally up to about 140°) to allow localized expansion as an ingested clot transits the section and is further compressed. The braid density pattern at a proximal end of the intermediate section 122 can be maintained in the most proximal braid section 123.

The braid 120 can have expansile sections with axial lengths 116 that are relatively long (for example, between approximately 5 mm and 10 mm) for clot management characteristics so that a longer section can ingest and compress more of a clot. Alternatively, the length 116 can be kept short (for example, between approximately 1 mm and 5 mm) for improved hoop strength and trackability. Braid angles of 110° or lower can provide radial expansion under compression, with lower angles offering greater expansion capability. Therefore, the length 116 of the expansion zones 121, 122 can be tailored by changing the distances at which angles of 125° or lower are maintained. Alternatively, the expansion zones 121, 122 can have a variable braid angle over at least a portion of the length 116.

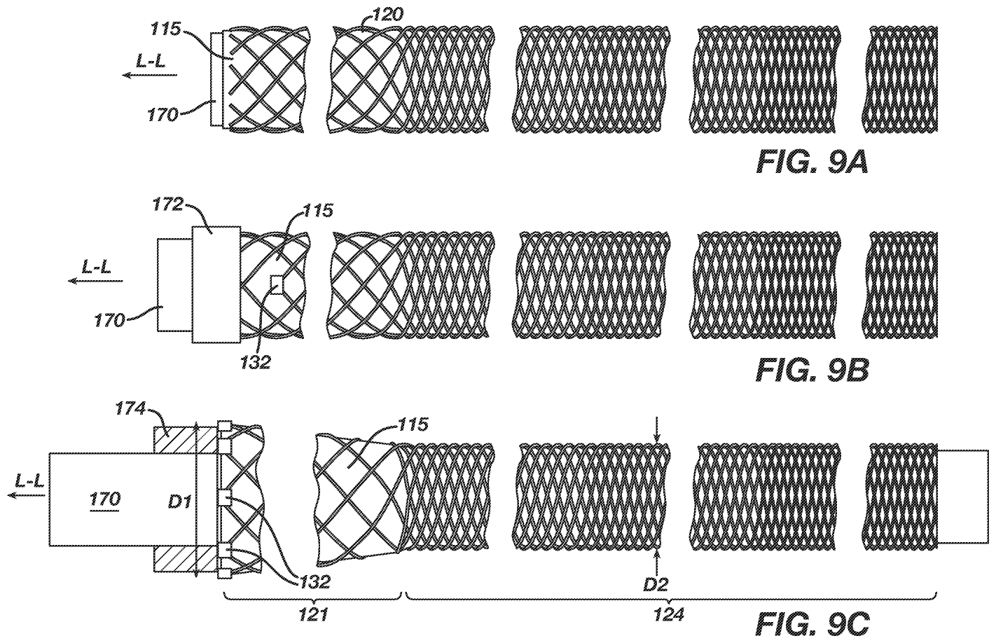

FIGS. 9A through 9C are illustrations of manufacturing steps of an example catheter. In FIG. 9A, braid 120 is woven directly on liner 115 and a spool 170. The braid 120 can be woven with sections of different PPI such as disclosed elsewhere herein (e.g. FIG. 8) or a variation thereof as understood by a person skilled in the pertinent art. In FIG. 9B, tape 172 is added to hold the braid 120 and braid ends are cut. Braid terminations 132 are added. The strands 140, 150 can be cut in pairs, and a sleeve 132, 232, 332, 432 or band 532 can be affixed to distal portions 143, 153 of the strands 140, 150 in the pair. In FIG. 9C, the distal segment 121 of the braid 120 and a distal portion of the liner 115 can be flared by positioning an inner hole of a funnel cone 174 over the spool 170. The funnel cone 174 is illustrated in cross-section in FIG. 9C for the sake of illustration. A tensile load can be applied to straighten the braid 120 before or after the braid terminations 132 are added as illustrated in FIG. 9B or before or after the distal portion is flared in FIG. 9C. Once flared, the distal segment 121 of the braid 120 can be annealed and/or heat set by laser or other methods as understood by a person skilled in the pertinent art. Preferably, the liner 115 is shielded from excess heat during annealing and/or heat setting.

Following the step illustrated in FIG. 9C, the outer jacket 160 and other features disclosed herein can be added to the catheter 100. The polymers of the jacket 160 and/or liner 115 can be reflowed. The spool 170 and funnel cone 174 can be removed.

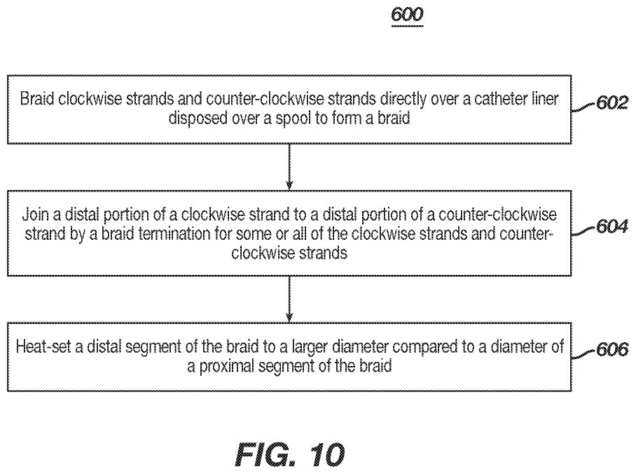

FIG. 10 is a flow diagram of a method 600 of constructing an example catheter. The resulting catheter can be configured similar to the catheter 100 disclosed elsewhere herein, variations thereof, and alternatives thereto as understood by a person skilled in the pertinent art.

At step 602 clockwise strands and counter-clockwise strands can be braided directly over a catheter liner disposed over a spool so that the strands form a braid. The strands can be configured similarly to strands 140, 150 disclosed elsewhere herein, variations thereof, and alternatives thereto as understood by a person skilled in the pertinent art. The strands can be braided to have a braid pattern similar to those disclosed elsewhere herein, variations thereof, and alternatives thereto as understood by a person skilled in the pertinent art. Braiding clockwise strands and counter-clockwise strands can be executed such that the braid has a greater braid pattern density on a proximal segment of the braid compared to a braid pattern density on the distal segment of the braid.

At step 604, a distal portion of a clockwise strand can be joined to a distal portion of a counter-clockwise strand by a braid termination for some or all of the clockwise strands and the counter-clockwise strands. The braid termination can be configured similarly to the braid terminations 130, 230, 330, 430, 530 disclosed elsewhere herein, variations thereof, and alternatives thereto as understood by a person skilled in the pertinent art. A sleeve can be crimped around the distal portion of the clockwise strand and the distal portion of the counter-clockwise strand. Additionally, or alternatively, a sleeve can be slid over the distal portion of the clockwise strand and the distal portion of the counter-clockwise strand. Distal portions of the strands can be aligned parallel and/or orthogonal to a longitudinal axis of the catheter distal portion. The distal portion of the clockwise strand can be joined to the distal portion of the counter-clockwise strand by the braid termination for some or all of the clockwise strands and counter-clockwise strands while the braid is directly over the catheter liner disposed over the spool.

At step 606, a distal section of the braid can be heat-set to a larger diameter compared to a diameter of a proximal segment of the braid. The distal segment can be heat-set to a funnel shape. The distal segment can be annealed. The distal segment can be heat-set to a shape using similar methodologies as disclosed herein to heat set the distal segment 121 disclosed elsewhere herein, variations thereof, and alternatives thereto as understood by a person skilled in the pertinent art. The shape of the distal segment can be similar to that of the distal segment 121 disclosed elsewhere herein, variations thereof, and alternatives thereto as understood by a person skilled in the pertinent art.

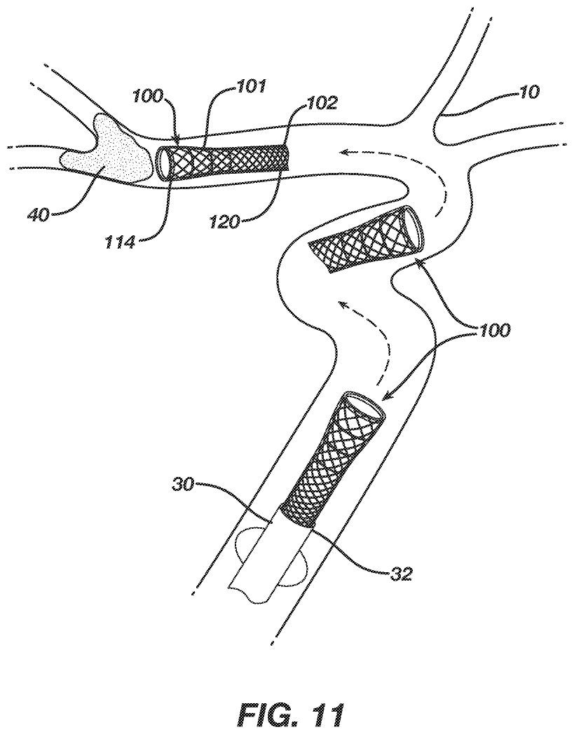

FIG. 11 is an illustration of a possible sequence for approaching an occlusive clot 40 using a large bore clot retrieval catheter 100 of the designs disclosed herein. The clot 40 can be approached with the catheter 100 collapsed within a guide sheath 30 or other outer catheter for delivery. When the vasculature 10 becomes too narrow and/or tortuous for further distal navigation with the guide sheath 30, the catheter 100 can be deployed for further independent travel distally. The catheter 100 can be highly flexible such that it is capable of navigating the M1, M2 or other tortuous regions of the neurovascular to reach an occlusive clot.

The clot retrieval catheter 100 can have a flexible elongate body 110 serving as a shaft with a large internal bore (which in some cases can be 0.090 inches or larger) and a distal tip section 101 having a collapsible supporting braided structure 120. The large bore helps the catheter to be delivered to a target site by a variety of methods. These can include over a guidewire, over a microcatheter, with a dilator/access tool, or by itself.

In most cases, the design of the collapsible funnel tip can be configured so that the catheter 100 can be delivered through (and retrieved back through) commonly sized outer sheaths and guides. For example, a standard 6Fr sheath/8Fr guide, would typically have an inner lumen of less than 0.090 inches. The tip can then be designed with a collapsed delivery outer diameter of approximately 0.086 inches. The tip can self-expand once advanced to an unconstrained position distal to the distal end 32 of the guide sheath 30, capable of reaching expanded outer diameters as large as approximately 0.132 inches. As the catheter can be delivered independently to a remote occlusion, the tip section 101 is ideally designed to be able to resist collapse from the forces of aspiration, has excellent lateral flexibility in both the expanded and collapsed states, an atraumatic profile to prevent snagging on bifurcations in vessels, and conformability to allow self-sizing should the tip need to be advanced through vessels with a diameter smaller than the tip and track past calcified plaque without dislodging it.

In describing example embodiments, terminology has been resorted to for the sake of clarity. As a result, not all possible combinations have been listed, and such variants are often apparent to those of skill in the art and are intended to be within the scope of the claims which follow. It is intended that each term contemplates its broadest meaning as understood by those skilled in the art and includes all technical equivalents that operate in a similar manner to accomplish a similar purpose without departing from the scope and spirit of the invention. It is also to be understood that the mention of one or more steps of a method does not preclude the presence of additional method steps or intervening method steps between those steps expressly identified. Similarly, some steps of a method can be performed in a different order than those described herein without departing from the scope of the disclosed technology.

What is claimed is:

1. A catheter comprising:
a braid extending through a distal section of the catheter and comprising clockwise strands and counter-clockwise strands;
a plurality of braid terminations each joining a distal portion of a clockwise strand to a distal portion of a counter-clockwise strand;
an inner liner disposed within the braid; and
an outer jacket disposed over the braid.

2. The catheter of claim 1, wherein an inner diameter of the distal section of the catheter is expandable when the distal section is unconstrained and compressible when the distal section is constrained.

3. The catheter of claim 1, wherein the plurality of braid terminations each comprise a sleeve over the distal portion of the clockwise strand and over the distal portion of the counter-clockwise strand.

4. The catheter of claim 3, wherein the sleeve comprises a crimped B-shape comprising a flat surface and two curved surfaces opposite the flat surface.

5. The catheter of claim 4, wherein the sleeve further comprises ends terminating the curved surfaces between the distal portion of the clockwise strand and the distal portion of the counter-clockwise strand.

6. The catheter of claim 3,
wherein the sleeve comprises a tubular shape comprising a lumen therethrough, and
wherein the distal portion of the clockwise strand and the distal portion of the counter-clockwise strand extend into the lumen.

7. The catheter of claim 6,
wherein the sleeve comprises an outer wall and an inner wall, and
wherein the outer wall is thicker than the inner wall.

8. The catheter of claim 1, wherein the distal portion of the clockwise strand and the distal portion of the counter-clockwise strand are each oriented parallel to a longitudinal axis defined by the distal section of the catheter.

9. The catheter of claim 1, wherein the distal portion of the clockwise strand and the distal portion of the counter-clockwise strand are each oriented orthogonal to a longitudinal axis defined by the distal section of the catheter.

10. The catheter of claim 1,
wherein the plurality of braid terminations comprise a band joining a first pair of braid distal portions, joining a second pair of braid distal portions, and extending between the first pair of braid distal portions and the second pair of braid distal portions, and
wherein each pair of distal portions comprises a distal portion of a clockwise strand and a distal portion of a counter-clockwise strand.

11. The catheter of claim 1, wherein the plurality of braid terminations each comprises radiopaque material.

12. The catheter of claim 1, wherein the braid has a greater braid pattern density in a proximal section of the catheter compared to a braid pattern density in the distal section of the catheter.

13. The catheter of claim 1,
wherein the clockwise strands and/or the counter-clockwise strands comprise radiopaque material, and
wherein the clockwise strands and/or the counter-clockwise strands comprise memory shape material.

14. The catheter of claim 1, wherein the plurality of braid terminations each comprise a laser weld.

15. The catheter of claim 1, wherein the plurality of braid terminations each comprise adhesive.

16. A method of constructing a catheter, the method comprising:
braiding clockwise strands and counter-clockwise strands directly over a catheter liner disposed over a spool to form a braid;
joining a distal portion of a clockwise strand to a distal portion of a counter-clockwise strand by a braid termination for some or all of the clockwise strands and counter-clockwise strands; and
heat-setting a distal segment of the braid to a larger diameter compared to a diameter of a proximal segment of the braid.

17. The method of claim 16, wherein joining the distal portion of the clockwise strand to the distal portion of the counter-clockwise strand by the braid termination for some or all of the clockwise strands and counter-clockwise strands further comprises crimping a sleeve around the distal portion of the clockwise strand and the distal portion of the counter-clockwise strand.

18. The method of claim 16, wherein joining the distal portion of the clockwise strand to the distal portion of the counter-clockwise strand by the braid termination for some or all of the clockwise strands and counter-clockwise strands further comprises sliding a sleeve over the distal portion of the clockwise strand and the distal portion of the counter-clockwise strand.

19. The method of claim 16, further comprising:
aligning the distal portion of the clockwise strand and the distal portion of the counter-clockwise strand parallel to a longitudinal axis of the catheter.

20. The method of claim 16, further comprising:
aligning the distal portion of the clockwise strand and the distal portion of the counter-clockwise strand orthogonal to a longitudinal axis of the catheter.
